(19) United States
(12) Patent Application Publication
(10) Pub. No.: US 2008/0275435 A1
(43) Pub. Date: Nov. 6, 2008

(54) Illuminated Laser Probe with Multiplied Area of Illumination

(75) Inventor: Timothy John Nadolski, Webster Grover, MO (US)

Correspondence Address: THOMPSON COBURN, LLP, ONE US BANK PLAZA, SUITE 3500, ST LOUIS, MO, 63101, US

(73) Assignee: Synergetics Inc., St. Charles, MO (US)

(21) Appl. No.: 11/913,436

(22) PCT Filed: May 4, 2006
(86) PCT No.: PCT/US2006/016783

§ 371 Date: Nov. 2, 2007

Related U.S. Application Data
(60) Provisional application No. 60/677,696, filed on May 4, 2005.

(51) Int. Cl. A61B 18/20

(52) U.S. Cl. 606/15

(57) ABSTRACT

A microsurgical laser probe has a handle and a tubular tip that projects from the handle, and three optic fibers that extend through the handle and the tip. One of the optic fibers is a laser optic fiber that is adapted to be connected to a source of laser light at one end of the laser optic fiber, and to transmit the laser light from the opposite end of the laser optic fiber at the distal end of the instrument tip. The other two optic fibers are illumination light optic fibers. Each illumination light optic fiber has one end that is adapted to be connected to a illumination light source, and an opposite end that projects the illumination light from the distal end of the instrument tip. By providing two laser light optic fibers in the instrument, the amount of illumination directed to the surgical site being accessed by the laser optic fiber is effectively doubled.

This patent application claims the benefit of provisional patent application No. 60/677,696, filed May 4, 2005.

### BACKGROUND OF THE INVENTION

(1) Field of the Invention

The present invention pertains to a microsurgical laser probe used primarily in ophthalmic surgery. The probe has a handle and a tubular tip that projects from the handle, and three optic fibers that extend through the handle and the tip.

One of the optic fibers is a laser optic fiber. The laser optic fiber is specifically adapted to be connected to a source of laser light at one end of the laser optic fiber, to conduct the laser light through the fiber, and to transmit the laser light from the opposite end of the laser optic fiber at the distal end of the instrument tip.

The other two optic fibers are illumination light optic fibers. Each illumination light optic fiber is specifically adapted to be connected to a illumination light source at one end of the illumination optic fiber, to conduct the illumination light through the illumination fiber, and to transmit the illumination light from the distal end of the instrument tip. By providing two illumination light optic fibers in the instrument, the amount of illumination directed to the surgical site being accessed by the laser optic fiber is effectively doubled.

(2) Description of the Related Art

In ophthalmic surgery, various different types of instruments are available for use by the surgeon to transmit laser energy to a surgical site in the interior of the eye. The typical microsurgical laser probe comprises a handle with a small cylindrical metal tip that projects from a distal end of the handle. A laser optic fiber, having a proximal end with a connector for coupling to a source of laser light, passes through the handle and the tip of the probe. The distal end of the laser optic fiber is positioned adjacent the distal end of the instrument tip. Connecting the laser light connector to a source of laser light and activating the source of laser light enables the surgeon to direct the laser light from the instrument tip to a surgical site.

Illumination probes are also often used in ophthalmic surgery. The illumination probe has a similar construction to the laser probe, except that the optic fiber extending through the illumination probe is specifically designed for transmission of illumination light, where the optic fiber that extends through the laser probe is specifically designed for the transmission of laser light. In the typical ophthalmic surgery procedure, an incision must be made in the eye to accommodate the tip of the laser probe, and a second incision must be made through the eye to accommodate the tip of the illumination probe. Furthermore, the illumination probe must be manipulated by the surgeon in order to direct the illuminating light to the desired surgical site while the surgeon simultaneously manipulates the laser probe to direct the laser light to the desired surgical site. The requirement that both of the surgical instruments be simultaneously manipulated to direct both illumination light and laser light to the surgical site adds complexity to an already complex surgical procedure.

### SUMMARY OF THE INVENTION

The present invention provides a laser probe designed primarily for ophthalmic surgery that overcomes the disadvantages associated with prior art laser probes by providing both a laser light transmitting optic fiber and an illumination light transmitting optic fiber in the same probe. Furthermore, the illumination function of the laser probe of the invention is multiplied by providing two illumination light transmitting optic fibers in the same instrument with the laser light transmitting optic fiber.

The instrument of the invention has a narrow, elongate handle that is designed to fit comfortably into the surgeon's hand. An elongate tubular tip of surgical steel projects from the handle.

A laser light transmitting optic fiber extends through both the handle and the tip. The proximal end of the laser light optic fiber is adapted for connection to a laser light source. The opposite distal end of the laser light fiber is positioned adjacent the instrument tip for transmitting laser light from the instrument to a surgical site.

A novel feature of the invention is provided by a pair of illumination light transmitting optic fibers that extend through the handle and the tip. Each illumination optic fiber has a proximal end that is adapted for connection to a separate source of illumination light. The opposite distal ends of the pair of illumination optic fibers are positioned adjacent the distal end of the tip. The illumination light transmitted from both illumination fiber distal ends provides a multiplied area of illumination at the surgical site being accessed by the laser light. In the preferred embodiment of the invention, to reduce the size of the instrument for use of the instrument in microscopic surgery, the laser light optic fiber and the pair of illumination light optic fibers are the only optic fibers that extend through the handle and the tip. Also in the preferred embodiment, the proximal ends of the two illumination light optic fibers are adapted for connection to two separate sources of illumination light. This effectively doubles the amount of illumination transmitted to the surgical site than was previously available using prior art ophthalmic surgery instruments.

### BRIEF DESCRIPTION OF THE DRAWING FIGURES

Features of the invention are set forth in the following detailed description of the preferred embodiment of the invention and in the following drawing figures wherein:

FIG. 1 is a cross section view of the surgical instrument of the invention;

FIG. 2 is an enlarged partial view of the portion of the instrument shown in circle 2 of FIG. 1; and,

FIG. 3 is an enlarged partial view of the portion of the instrument shown in circle 3 of FIG. 1.

### DETAILED DESCRIPTION OF A PREFERRED EMBODIMENT

The surgical instrument of the invention is primarily intended to provide illumination light and laser light in laser eye surgery. However, the instrument may be used in other types of surgery. In addition, instead of laser light, the instrument could be provided with a surgical device that provides aspiration to a surgical site, or the instrument could be provided with a bipolar cautery device, or some other type of surgical device. The instrument is designed as a disposable instrument, but alternatively, the instrument could be a reusable instrument that is sterilized after each use.

The instrument has an elongate, narrow handle or hand piece 12 that has opposite proximal 14 and distal 16 ends. The handle 12 is dimensioned to a size similar to that of a pencil to fit comfortably in a surgeon's hand and to be easily manually manipulated by the surgeon's hand. A hollow interior bore 18 extends through the center of the handle 12 from the handle proximal end 14 to the handle distal end 16.

An elongate, tubular tip 32 projects from the handle distal end 16. The tip is preferably constructed of surgical steel, and has the general construction of a hypodermic needle. The tubular tip 32 has an interior bore that extends through the tip from a proximal end 36 of the tip to a distal end 38 of the tip. The tip proximal end 36 is received in the handle interior bore 18 at the handle distal end 16 and is secured stationary in place by adhesives 40 or other equivalent means. In alternative embodiments of the instrument, the tip 32 can be curved along a portion of its length.

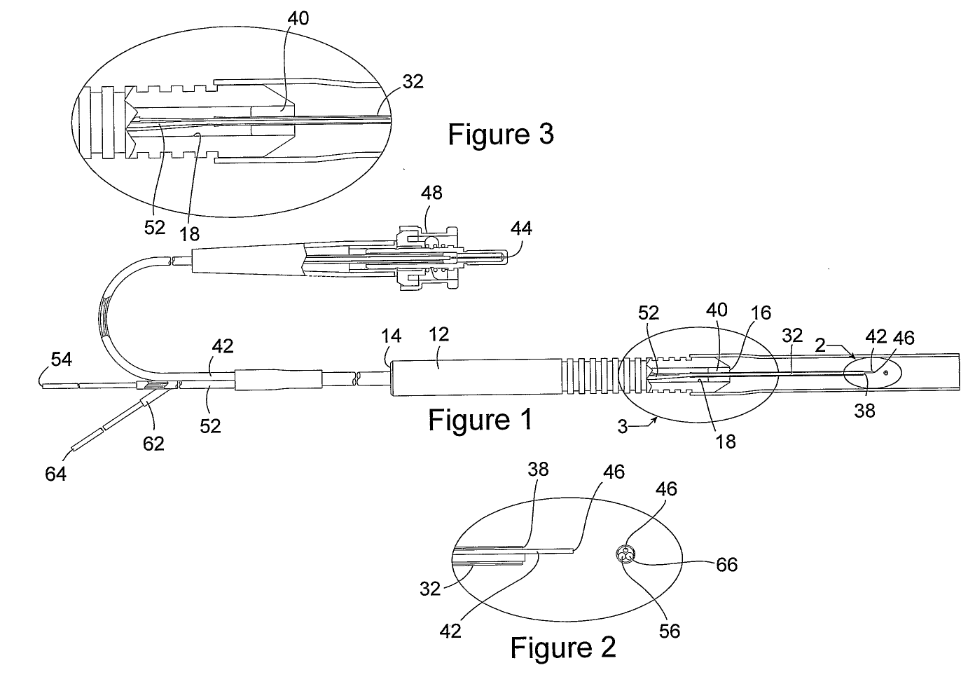

A laser optic fiber 42 is one example of a surgical device that can be used in the instrument of the invention. The laser optic fiber 42 is specifically designed to conduct and transmit laser light. As stated earlier, other types of surgical devices could be used, and the instrument should not be viewed as only usable with a laser optic fiber. For example, in place of the laser optic fiber, the instrument could comprise an aspiration tube, or a bipolar cautery device, or some other type of surgical device. The laser optic fiber 42 has an elongate, continuous length with opposite proximal 44 and distal 46 ends. The optic fiber extends through the handle 12 and through the tip 32 to the fiber distal end 46. A laser connector 48 is provided at the laser optic fiber proximal end 44. The laser connector 48 is adapted for connecting the laser optic fiber 42 to a separate laser light source 50 that is represented schematically in FIG. 1. Laser light sources of this type are known in the art and produce laser wavelength light for laser surgery for transmission by an optic fiber. With the connector 48 connected to the laser light source 50, on operation of the light source 50 laser light is transmitted through the laser optic fiber to the laser optic fiber distal end 46. The laser optic fiber 42 extends through the handle interior bore 18 and through the tip interior bore 34 to the laser optic fiber distal end 46 that is extended a set distance outwardly from the tip distal end 38. A portion of the laser optic fiber 42 is secured to the interior of the tip 32 by an epoxy or other equivalent means.

A first illumination optic fiber 52 having a continuous length with opposite proximal 54 and distal 56 ends extends through the handle 12 and through the tip 32. The illumination optic fiber 52 is specifically designed to conduct and transmit illuminating light. The illumination optic fiber proximal end 54 is adapted to be connected to a first illumination light source 58 that is represented schematically in FIG. 1. Illumination light sources of this type are known in the art and produce illumination wavelength light for transmission by an optic fiber. With the illumination optic fiber proximal end 54 connected to the first light source 58, on operation of the light source 58 illumination light is transmitted through the illumination optic fiber 52 to the illumination optic fiber distal end 56. The illumination optic fiber distal end 56 is positioned adjacent the tip distal end 38. The spacing of the first illumination optic fiber distal end 56 back from the laser optic fiber distal end 46 enables light projected from the first illumination optic fiber distal end 56 to illuminate a large field around the surgical site adjacent the laser optic fiber distal end 46. As shown in FIG. 2, the illumination optic fiber distal end 56 has a larger surface area than the laser fiber distal end 46 to provide increased illumination of the surgical site. The illumination optic fiber 52 is secured stationary in the interior of the tip 32 and in the interior of the handle 12 by adhesives or other equivalent means.

A second illumination optic fiber 62, entirely separate from the first illumination optic fiber 52, also extends through the handle 12 and through the tip 32. The second illumination optic fiber 62 has a length with opposite proximal 64 and distal 66 ends. The second illumination optic fiber proximal end 64 is adapted to be connected to an entirely separate, second illumination light source 68, represented schematically in FIG. 1. The second illumination light source connected to the second illumination optic fiber 62 transmits additional illumination light through the second illumination optic fiber 62 to the second illumination optic fiber distal end 66. The second illumination optic fiber distal end 66 is positioned adjacent the tip distal end 38 and adjacent the first illumination optic fiber distal end 56. As shown in FIG. 2, the first and second illumination optic fibers 52, 62 have substantially the same cross-sectional areas, and thereby transmit twice as much light as that transmitted by a single optic fiber of the same cross-sectional area. The cross-sectional areas of each illumination fiber 52, 62 is larger than the cross-sectional area of the laser optic fiber 42. The spacing of the first and second illumination optic fiber distal ends 56, 66 back from the laser optic fiber distal end 46 enables the multiplied light projected from the first and second illumination optic fiber distal ends 56, 66 to provide multiplied illumination to a large field around the surgical site adjacent the laser optic fiber distal end 46. The second illumination optic fiber 62 is also secured stationary in the interior of the tip 32 and in the interior of the handle 12 by adhesives or other equivalent means.

In other embodiments of the surgical probe, the positions of the optic fiber distal ends can be varied. For example, the laser optic fiber distal end 46 and the illumination optic fiber distal ends 56, 66 could all be positioned adjacent or flush with each other. These distal ends of the optic fibers could also be positioned adjacent or flush with the tip distal end 38, or could all be extended outwardly from the tip distal end. Furthermore, the optic fiber distal ends 46, 56, 66 could be positioned at staggered positions relative to each other. The distal ends of the optic fibers 46, 56, 66 could each have different configurations from the flat end surfaces shown in the drawings. For example, the distal end surfaces could have a cone shape, a bevel shape, a bullet shape, or any of various other shapes.

In use of the surgical instrument, the laser optic fiber proximal end 44 is connected to a source of laser light 50 and the first and second illumination optic fiber proximal ends 54, 64 are connected to separate sources of illumination light 58, 68. In alternate embodiments, for example where the surgical device is an aspiration tube, the proximal end of the aspiration tube would be connected to a source of aspiration. Where the surgical instrument is a bipolar cautery device, the proximal end of the cautery device would be connected with a power source for the device. Also, a single source of illumination light with two separate optic fiber connections could be used.

The tip 32 is then inserted through a cannula positioned in an incision in the eye, or is inserted directly through the incision. The tip is positioned in the eye with the tip distal end 38 positioned relative to the surgical site to provide the desired area of illumination from the illumination light transmitted from the first and second illumination optic fiber distal ends 56, 66.

With the desired area of the surgical site illuminated, the laser optic fiber distal end 46 is positioned at a desired position relative to the surgical site to begin the surgical procedure.

Thus, as discussed above, the surgical instrument of the present invention provides a source of laser light for performing surgery and a source of illumination where the illumination provided to the area of the surgical site is multiplied.

Although a specific embodiment of the invention has been described herein, it should be understood that other modifications and variations may be made to the invention without departing from the intended scope of protection provided by the following claims.

1) (canceled)

2) A surgical instrument that provides both illumination light and laser light to a surgical site, the surgical instrument comprising:
a manually manipulatable handle;
a tubular tip secured to the handle, the tip projecting from the handle to a distal end of the tip;
a first illumination optic fiber having a length with opposite proximal and distal ends, the first illumination optic fiber extending through the handle and the tip to the first illumination optic fiber distal end positioned adjacent the tip distal end, the first illumination optic fiber being secured stationary relative to the tip;
a laser optic fiber having a length with opposite proximal and distal ends, the laser optic fiber extending through the handle and the tip to the laser optic fiber distal end positioned adjacent the tip distal end;
a second illumination optic fiber having a length with opposite proximal and distal ends, the second illumination optic fiber extending through the handle and the tip to the second illumination optic fiber distal end positioned adjacent the tip distal end, the second illumination optic fiber being secured stationary relative to the tip; and,
the first and second illumination optic fibers having proximal ends that are connectable to two separate sources of illumination light.

3) The surgical instrument of claim 2, further comprising:
a first illumination light source, the first illumination optic fiber proximal end being connected to the first illumination light source; and,
a second illumination light source, the second illumination optic fiber proximal end being connected to the second illumination light source.

4) The surgical instrument of claim 3, further comprising:
the first illumination light source being separate from the second illumination light source.

5) The surgical instrument of claim 3, further comprising:
a laser light source, the laser optic fiber proximal end being connected to the laser light source.

6) The surgical instrument of claim 2, further comprising:
the first illumination optic fiber distal end having a cross-sectional area that is larger than a cross-sectional area of the laser optic fiber distal end; and,
the second illumination optic fiber distal end having a cross-sectional area that is larger than the cross-sectional area of the laser optic fiber distal end.

7) The surgical instrument of claim 2, further comprising:
the laser optic fiber and the first and second illumination optic fibers being the only optic fibers that extend through the tip.

8) The surgical instrument of claim 2, further comprising:
the first and second illumination optic fiber proximal ends being movable relative to each other.

9) The surgical instrument of claim 2, further comprising:
the first and second illumination optic fiber distal ends being substantially positioned in a single plane.

10) The surgical instrument of claim 2, further comprising:
a portion of the laser optic fiber adjacent the laser optic fiber distal end extending outwardly from the first and second illumination optic fiber distal ends.

11) The surgical instrument of claim 10, further comprising:
the portion of the laser optic fiber adjacent the laser optic fiber distal end extending outwardly from the tip distal end.

12) A surgical instrument that provides both illumination light and laser light to a surgical site, the surgical instrument comprising:
an elongate handle having opposite proximal and distal ends, the handle having an interior bore with an interior surface extending through the handle from the handle proximal end to the handle distal end, and the handle having an exterior surface;
an elongate tubular tip having opposite proximal and distal ends, the tip having an interior bore with an interior surface extending through the tip from the tip proximal end to the tip distal end, the tip proximal end being secured to the handle at the handle distal end;
a first illumination optic fiber having a length with opposite proximal and distal ends, the first illumination optic fiber proximal end being adapted to be attached to a separate first illumination light source to transmit illumination light through the first illumination optic fiber to the first illumination optic fiber distal end, the first illumination optic fiber extending through the handle interior bore and through the tip interior bore to the first illumination optic fiber distal end positioned adjacent the tip distal end, the first illumination optic fiber being secured stationary relative to the handle and the tip;
a laser optic fiber having a length with opposite proximal and distal ends, the laser optic fiber proximal end being adapted to be attached to a separate laser light source to transmit laser light through the laser optic fiber to the laser optic fiber distal end, the laser optic fiber extending through the handle interior bore and through the tip interior bore to the laser optic fiber distal end positioned adjacent the tip distal end; and,
a second illumination optic fiber having a length with opposite proximal and distal ends, the second illumination optic fiber proximal end being separate from the first illumination optic fiber proximal end and being adapted to be attached to a separate second illumination light source to transmit illumination light through the second illumination optic fiber to the second illumination optic fiber distal end, the second illumination optic fiber extending through the handle interior bore and through the tip interior bore to the second illumination optic fiber distal end positioned adjacent the tip distal end, the second illumination optic fiber being secured stationary relative to the handle and the tip.

13) The surgical instrument of claim 12, further comprising:
the first and second illumination optic fiber proximal ends being connectable to two separate illumination light sources.

14) The surgical instrument of claim 12, further comprising:
the first and second illumination optic fiber proximal ends being movable relative to each other.

15) The surgical instrument of claim 12, further comprising:
the first and second illumination optic fiber distal ends being substantially positioned in a single plane.

16) The surgical instrument of claim 12, further comprising:
a portion of the laser optic fiber adjacent the laser optic fiber distal end extending outwardly from the first and second illumination optic fiber distal ends.

17) The surgical instrument of claim 16, further comprising:
the portion of the laser optic fiber adjacent the laser optic fiber distal end extending outwardly from the tip distal end.

18) The surgical instrument of claim 12, further comprising:
the laser optic fiber distal end having a cross-sectional area that is smaller than a cross-sectional area of the first illumination optic fiber distal end and is smaller than a cross-sectional area of the second illumination optic fiber distal end.

19) The surgical instrument of claim 12, further comprising:
the laser optic fiber and the first and second illumination optic fibers being the only optic fibers that extend through the tip.

20) A surgical instrument that provides both illumination light and laser light to a surgical site, the surgical instrument comprising:
an elongate handle having opposite proximal and distal ends, the handle having an interior bore with an interior surface extending through the handle from the handle proximal end to the handle distal end, and the handle having an exterior surface;
an elongate tubular tip having opposite proximal and distal ends, the tip having an interior bore with an interior surface extending through the tip from the tip proximal end to the tip distal end, the tip proximal end being secured to the handle at the handle distal end;
a first illumination optic fiber having a length with opposite proximal and distal ends, the first illumination optic fiber proximal end being adapted to be attached to a separate first illumination light source to transmit illumination light through the first illumination optic fiber to the first illumination optic fiber distal end, the first illumination optic fiber extending through the handle interior bore and through the tip interior bore to the first illumination optic fiber distal end positioned adjacent the tip distal end, the first illumination optic fiber being secured stationary relative to the handle and the tip;
a laser optic fiber having a length with opposite proximal and distal ends, the laser optic fiber proximal end being adapted to be attached to a separate laser light source to transmit laser light through the laser optic fiber to the laser optic fiber distal end, the laser optic fiber extending through the handle interior bore and through the tip interior bore to the laser optic fiber distal end positioned adjacent the tip distal end; and,
a second illumination optic fiber having a length with opposite proximal and distal ends, the second illumination optic fiber proximal end being separate from and movable relative to the first illumination optic fiber proximal end and be adapted to be attached to a separate second illumination light source to transmit illumination light through the second illumination optic fiber to the second illumination optic fiber distal end, the second illumination optic fiber extending through the handle interior bore and through the tip interior bore to the second illumination optic fiber distal end positioned adjacent the tip distal end, the second illumination optic fiber being secured stationary relative to the handle and the tip.
